(19) United States
(12) Patent Application Publication
(10) Pub. No.: US 2014/0137295 A1
(43) Pub. Date: May 15, 2014

(54) USE OF INVERTASE SILENCING IN POTATO TO MINIMIZE CHIP AND FRENCH FRY LOSSES FROM ZEBRA CHIP AND SUGAR ENDS

(71) Applicant: J.R. Simplot Company, Boise, ID (US)

(72) Inventors: Craig RICHAEL, Boise, ID (US); Jingsong Ye, Boise, ID (US); Caius Rommens, Boise, ID (US)

(73) Assignee: J.R. Simplot Company, Boise, ID (US)

(21) Appl. No.: 14/076,471

(22) Filed: Nov. 11, 2013

Related U.S. Application Data
(60) Provisional application No. 61/724,632, filed on Nov. 9, 2012.
(60) Provisional application No. 61/783,390, filed on Mar. 14, 2013.

(51) Int. Cl. C12N 15/113

(52) U.S. Cl. CPC C12N 15/113
USPC 800/286

(57) ABSTRACT

The present invention provides a convenient method for producing potato products such as chips and French fries that have lower incidence of sugar ends and less off-color development due to infection from the zebra chip pathogen.

### CROSS-REFERENCE TO RELATED APPLICATIONS

This application claims priority to U.S. Provisional Application Ser. No. 61/724,632, filed Nov. 9, 2012, and U.S. Provisional Application Ser. No. 61/783,390, filed Mar. 14, 2013, each of which is herein incorporated by reference in its entirety.

### FIELD OF THE INVENTION

The present invention provides convenient methods for producing potato products including chips and French fries that have lower incidence of sugar ends and/or less off-color development due to infection from the zebra chip pathogen.

### BACKGROUND

Potato (Solanum tuberosum) is the third most important food crop in the world. It is used for human consumption, animal feed and as a source of starch and alcohol. Over two thirds of the global production is eaten directly by humans with much of the rest being fed to animals or used to produce starch. The annual diet of an average global citizen in the first decade of the 21st century included about 33 kg (or 73 lb) of potato.

During every growing season potato plants are subjected to a variety of biotic and abiotic stresses that impact plant health, yields and final tuber quality. Poor tuber quality due to the combined effect of environmental and cultural practices in the field can be visualized in the final products, such as the French fry or potato chip. Suboptimal growing years and poor cultural practices result in an obvious increase of internal tuber disorders such as brown spot, hollow heart, internal necrosis, vascular discoloration, Zebra Chip and sugar ends. Finished fry products with these disorders must be discarded, constituting an economic loss to the processor. Some of the economic burden is passed on the grower in the form of contract penalties or to the consumer in the form of higher prices for the product.

Thus, there is a continuing need for improvement of potato tuber quality, which the present invention addresses.

### SUMMARY OF THE INVENTION

The present invention provides methods of minimizing the frequency of sugar ends in potato tuber or products made from said potato tuber, wherein the frequency of sugar ends in the potato tuber is reduced in comparison to a control potato tuber. In some embodiments, the methods comprise disrupting the vacuolar invertase enzyme activity in said potato tuber.

The present invention also provides methods of minimizing the symptoms of Zebra chip in potato tuber or products made from said potato tuber, wherein the symptoms of Zebra chip in the potato tuber is reduced in comparison to a control potato tuber. In some embodiments, the methods comprise disrupting the vacuolar invertase enzyme activity in said potato tuber,

The vacuolar invertase enzyme activity can be disrupted by any suitable method. In some embodiments, vacuolar invertase enzyme activity is disrupted by introducing one or more nucleotide changes of the vacuolar invertase gene encoding the vacuolar invertase enzyme into the potato tuber. In some embodiments, the nucleotide changes happen naturally, or are created artificially by any suitable methods. In some embodiments, the vacuolar invertase enzyme activity is disrupted by introducing one or more inhibitory nucleotide sequences. In some embodiments, the inhibitory nucleotide sequence is selected from the group consisting of antisense RNA sequences, dsRNAi sequences, and inverted repeats.

In some embodiments, the inhibitory nucleotide is operably linked to a plant promoter. In some embodiments, the plant promoter is selected from the group consisting of constitutive promoters, non-constitutive promoters, inducible promoters, tissue specific promoters, and cell-type specific promoters.

In some embodiments, the tissue specific promoter is a tuber-specific promoter. In some embodiments, the tuber-specific promoter is a promoter associated with an ADP glucose pyrophosphorylase gene. In some embodiments, the tuber-specific promoter comprises the nucleic acid sequence SEQ ID NO: 6, or any functional variants therefore or functional fragments thereof.

In some embodiments, the inhibitory nucleotide sequence is an inverted repeat sequence. In some embodiments, the inverted repeat is derived from SEQ ID NO: 5. In some embodiments, the inverted repeat comprises at least one sense sequence and at least one anti-sense sequence which share at least 80%, 85%, 90%, 95% 99% or more similarity to certain part or parts of SEQ ID NO: 5 or its reverse complementary sequence. In some embodiments, the inverted repeat comprises at least one sense sequence and at least one anti-sense sequence which can hybridize with SEQ ID NO: 5 or its reverse complementary sequence.

In some embodiments, the inverted repeat comprises a sense sequence corresponding to +53 to +733 of SEQ ID NO: 5. In some embodiments, the inverted repeat comprises an anti-sense sequence corresponding to +552 to +49 of SEQ ID NO: 5.

The present invention also provides methods for producing a transgenic plant that does not produce tubers with sugar ends under conditions in the field normally conducive to the induction of sugar ends, and methods of using invertase silencing to minimize the symptoms of Zebra chip or to lower the frequency of sugar ends.

In some embodiments, the methods of the present invention comprise expressing a gene silencing cassette in a potato plant. In some embodiments, the cassette comprises a sense sequence and an antisense sequence oriented as an inverted repeat. In some embodiments, the sense sequence has 100% identity to SEQ ID NO: 5. In some embodiments, the antisense sequence is a full length or partial reverse and complement sequence of the sense sequence. In some embodiments, the sense sequence and the antisense sequence is separated by a spacer. In some embodiments, the expression cassette comprises a tuber-specific promoter. In some embodiments, the tuber-specific promoter is operably linked to the sense and the antisense sequences. In some embodiments, the expression of cassette down-regulates the expression of at least one endogenous invertase gene thereby minimizing the frequency of sugar ends in potato tuber or products made from said potato tuber, and/or minimizing the symptoms of Zebra chip in potato tuber or products made from said potato tuber. In some embodiments, the sense sequence is 100% identical to the full length or partial sequence of SEQ ID NO: 5. In some embodiments, the antisense sequence is 100% identical to the reverse and complement sequence of the sense sequence. For example, the sense sequence can be SEQ ID NO: 3, and the antisense sequence can be SEQ ID NO: 21. In some embodiments, the antisense sequence is not 100% identical to, but partially overlapped with the reverse and complement sequence of the sense sequence, for example, the sense sequence can be SEQ ID NO: 3, and the antisense sequence can be SEQ ID NO: 4.

The methods of present invention are not expected, because the gene efficacy is strictly associated with cold temperature induction previously (Klann et al., Plant Physiol 1993; Bethke and Jiang, Plant Physiol 2010; Ye et al. J. Agric Food Chem 2010).

### BRIEF DESCRIPTION OF THE FIGURES

FIG. 1. Sugar ends (SE) are apparent on nearly half of the ‘Ranger’ (A) and empty vector (B) control fries. Lines 1632-1 (C) and 1632-4 (D) are silenced for invertase and show no SE when grown and fried under the same conditions as the controls.

FIG. 2. Severe (A) and mild (B) Zebra chip symptoms on fresh potato slices. Severe symptoms of tissue necrosis throughout the tuber flesh are apparent on tubers infected 35 and 28 days before harvest (dbh). Less necrosis is apparent on mildly infected tuber slices at 21 dbh. Little or no necrosis is seen on tubers 14 and 7 dbh.

FIG. 3. Chip samples from a ‘Ranger’ control (left side) and an invertase-silenced line 1632-1 (right side) at (A) 35 days before harvest (dbh); (B) 28 dbh; (C) 21 dbh; (D) 14 dbh; and (E) 7 dbh. Chips were made from slices of 6-8 tubers and fried at 375° F. for 3 minutes. A final 2% moisture content was achieved.

FIG. 4. Silencing polyphenol oxidase (Ppo) eliminates the oxidative darkening of zebra chip infected tubers. Polyphenol oxidase action in uninfected cv. ‘Atlantic’ tubers (A) converts a colorless catechol substrate to the dark precipitate on the cut tuber surface. Neither uninfected (B) nor infected (C) Ppo-silenced tubers show the darkening. Three different tubers are shown. Photo taken 15 minutes after a 0.4 M catechol solution was applied over the cut tuber surface.

FIG. 5. Northerns demonstrate silencing of invertase (A) and Ppo (B). Ethidium bromide stained RNA gel below each Northern for loading reference. Total RNA (20 μg) was isolated from greenhouse-grown tubers. Tuber tissues of intragenic events and controls and hybridized with the Inv (A) and Ppo (B) probe.

### DESCRIPTION OF THE TEXT FILE SUBMITTED ELECTRONICALLY

The contents of the text file submitted electronically are incorporated herein by reference in their entirety: A computer readable format copy of the Sequence Listing (filename: JRSI00202US_ST25.txt, date recorded: Nov. 8, 2013, file size 20 kilobytes).

### DEFINITIONS

As used herein, the verb “comprise” as is used in this description and in the claims and its conjugations are used in its non-limiting sense to mean that items following the word are included, but items not specifically mentioned are not excluded.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

As used herein, the term “plant” refers to any living organism belonging to the kingdom Plantae (i.e., any genus/species in the Plant Kingdom). This includes familiar organisms such as but not limited to trees, herbs, bushes, grasses, vines, ferns, mosses and green algae. The term refers to both monocotyledonous plants, also called monocots, and dicotyledonous plants, also called dicots. For example, in some embodiments, the plant is a species in the Solanum genus, such as S. tuberosum S. stenotomum, S. phureja, S. goniocalyx, S. ajanhuiri. S. chaucha, S. juzepczukii, and S. curtilobum. In some embodiments, the plant is a potato variety of the S. tuberosum species.

As used herein, the term “plant part” refers to any part of a plant including but not limited to the shoot, root, stem, axillary buds, seeds, stipules, leaves, petals, flowers, ovules, bracts, branches, petioles, node, internodes, bark, pubescence, tillers, rhizomes, fronds, blades, pollen, stamen, microtubers, and the like.

As used herein, the term “germplasm” refers to the genetic material with its specific molecular and chemical makeup that comprises the physical foundation of the hereditary qualities of an organism.

As used herein, the phrase “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

As used herein, the term “offspring” refers to any plant resulting as progeny from a vegetative or sexual reproduction from one or more parent plants or descendants thereof. For instance an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

As used herein, a “plant promoter” is a promoter capable of initiating transcription in plant cells whether or not its origin is a plant cell.

As used herein, the “stringent hybridization conditions” comprise hybridization overnight (12-24 hrs) at 42° C. in the presence of 50% formamide, followed by washing, or 5×SSC at about 65° C. for about 12 to about 24 hours, followed by washing in 0.1×SSC at 65° C. for about one hour.

As used herein, a “constitutive promoter” is a promoter which is active under most conditions and/or during most development stages. There are several advantages to using constitutive promoters in expression vectors used in plant biotechnology, such as: high level of production of proteins used to select transgenic cells or plants; high level of expression of reporter proteins or scorable markers, allowing easy detection and quantification; high level of production of a transcription factor that is part of a regulatory transcription system; production of compounds that requires ubiquitous activity in the plant; and production of compounds that are required during all stages of plant development. Non-limiting exemplary constitutive promoters include, CaMV 35S promoter, opine promoters, ubiquitin promoter, actin promoter, alcohol dehydrogenase promoter, etc.

As used herein, a “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues, such as stems, leaves, roots, or seeds.

As used herein, “inducible” or “repressible” promoter is a promoter which is under chemical or environmental factors control. Examples of environmental conditions that may effect transcription by inducible promoters include anaerobic conditions, or certain chemicals, or the presence of light.

As used herein, a “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related plant species to achieve efficient and reliable expression of transgenes in particular tissues. This is one of the main reasons for the large amount of tissue-specific promoters isolated from particular plants and tissues found in both scientific and patent literature. Non-limiting examples of tissue specific promoters include, tuber-specific promoters, leaf-specific promoters, root-specific promoters, flower-specific promoters, seed-specific promoters, meristem-specific promoters, etc.

As used herein, a “cell type specific” promoter is a promoter that primarily drives expression in certain cell types in one or more organs.

As used herein, the term “variety” refers to a subdivision of a species, consisting of a group of individuals within the species that are distinct in form or function from other similar arrays of individuals.

As used herein, the term “genotype” refers to the genetic makeup of an individual cell, cell culture, tissue, organism (e.g., a plant), or group of organisms.

As used herein, the term “clone” refers to a cell, group of cells, a part, tissue, organism (e.g., a plant), or group of organisms that is descended or derived from and genetically identical or substantially identical to a single precursor. In some embodiments, the clone is produced in a process comprising at least one asexual step.

As used herein, the term “hybrid” refers to any individual cell, tissue or plant resulting from a cross between parents that differ in one or more genes.

As used herein, the term “inbred” or “inbred line” refers to a relatively true-breeding strain.

As used herein, the term “population” means a genetically homogeneous or heterogeneous collection of plants sharing a common genetic derivation.

As used herein, the term “variety” or “cultivar” means a group of similar plants that by structural features and performance can be identified from other varieties within the same species. The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

As used herein, the phrase “sugar ends” refers to a physiological disorder of tubers resulting from sugar accumulation to high levels at one end of the tuber, usually at the stolon end. French fries from tubers with sugar ends have dark brown ends, an undesirable processing defect.

As used herein, the term “Zebra chip” refers to a disease of potato caused by the pathogen Candidatus Liberibacter solanacearum, vectored by the potato psyllid Bactericera cockerelli. Chips and French fries from Zebra chip-infected potatoes have patterns of alternating brown and lighter brown color that usually renders them unmarketable.

### DETAILED DESCRIPTION

### Potato

There are about five thousand potato varieties worldwide. Three thousand of them are found in the Andes alone, mainly in Peru, Bolivia, Ecuador, Chile, and Colombia. They belong to eight or nine species, depending on the taxonomic school. Apart from the five thousand cultivated varieties, there are about 200 wild species and subspecies, many of which can be cross-bred with cultivated varieties, which has been done repeatedly to transfer resistances to certain pests and diseases from the gene pool of wild species to the gene pool of cultivated potato species.

The major species grown worldwide is Solanum tuberosum (a tetraploid with 48 chromosomes), and modern varieties of this species are the most widely cultivated. There are also four diploid species (with 24 chromosomes): S. stenotomum, S. phureja, S. goniocalyx, and S. ajanhuiri. There are two triploid species (with 36 chromosomes): S. chaucha and S. juzepczukii. There is one pentaploid cultivated species (with 60 chromosomes): S. curtilobum. There are two major subspecies of Solanum tuberosum: andigena, or Andean; and tuberosum, or Chilean. The Andean potato is adapted to the short-day conditions prevalent in the mountainous equatorial and tropical regions where it originated. The Chilean potato, native to the ChiloéArchipelago, is adapted to the long-day conditions prevalent in the higher latitude region of southern Chile.

Potatoes yield abundantly and adapt readily to diverse climates as long as the climate is cool and moist enough for the plants to gather sufficient water from the soil to form the starchy tubers. Potatoes do not keep very well in storage and are vulnerable to molds that feed on the stored tubers, quickly turning them rotten. By contrast, grain can be stored for several years without much risk of rotting.

Potato contains vitamins and minerals, as well as an assortment of phytochemicals, such as carotenoids and natural phenols. Chlorogenic acid constitutes up to 90% of the potato tuber natural phenols. Others found in potatoes are 4-O-caffeoylquinic acid (crypto-chlorogenic acid), 5-O-caffeoylquinic (neo-chlorogenic acid), 3,4-dicaffeoylquinic and 3,5-dicaffeoylquinic acids.[58] A medium-size 150 g (5.3 oz) potato with the skin provides 27 mg of vitamin C (45% of the Daily Value (DV)), 620 mg of potassium (18% of DV), 0.2 mg vitamin B6 (10% of DV) and trace amounts of thiamin, riboflavin, folate, niacin, magnesium, phosphorus, iron, and zinc. The fiber content of a potato with skin (2 g) is equivalent to that of many whole grain breads, pastas, and cereals.

In terms of nutrition, the potato is best known for its carbohydrate content (approximately 26 grams in a medium potato). The predominant form of this carbohydrate is starch. A small but significant portion of this starch is resistant to digestion by enzymes in the stomach and small intestine, and so reaches the large intestine essentially intact. This resistant starch is considered to have similar physiological effects and health benefits as fiber: It provides bulk, offers protection against colon cancer, improves glucose tolerance and insulin sensitivity, lowers plasma cholesterol and triglyceride concentrations, increases satiety, and possibly even reduces fat storage. The amount of resistant starch in potatoes depends much on preparation methods. Cooking and then cooling potatoes significantly increases resistant starch. For example, cooked potato starch contains about 7% resistant starch, which increases to about 13% upon cooling.

Potato has been bred into many standard or well-known varieties, each of which has particular agricultural or culinary attributes. In general, varieties are categorized into a few main groups, such as russets, reds, whites, yellows (also called Yukons) and purples—based on common characteristics. For culinary purposes, varieties are often described in terms of their waxiness. Floury, or mealy (baking) potatoes have more starch (20-22%) than waxy (boiling) potatoes (16-18%). The distinction may also arise from variation in the comparative ratio of amylose and amylopectin. In some embodiments, the potato variety of the present invention is a White Rounds potato variety, a Red Rounds potato variety, or a Russet potato variety.

In some embodiments, the potato is a variety deposited in the International Potato Center based in Lima, Peru, which holds an ISO-accredited collection of potato germplasm. The international Potato Genome Sequencing Consortium announced in 2009 that they had achieved a draft sequence of the potato genome. The potato genome contains 12 chromosomes and 860 million base pairs making it a medium-sized plant genome. More than 99 percent of all current varieties of potatoes currently grown are direct descendants of a subspecies that once grew in the lowlands of south-central Chile. In some other embodiments, the potato is a variety included in the European Cultivated Potato Databased (ECPD), the Potato Association of America, the Cornell Potato Varieties List, the Canadian Registry of Potato Varieties, the UPOV potato varieties collection, The British Potato Variety Database, International Potato Center, Potato Variety Management Institute, United States Potato GenBank, North Carolina State University Potato Variety Database, Texas A&M Potato Breeding & Variety Development Program, Michigan State University Potato Breeding and Genetics Program, and North American Potato Variety Inventory etc.

Exemplary potato varieties for which the present invention include, but are not limited to, Ranger Russet, Burbank, Innovator, Atlantic, Umatilla Russet, Adirondack Blue, Adirondack Red, Agata, Almond, Apline, Alturas, Amandine, Annabelle, Anya, Arran Victory, Avalanche, Bamberg, Bannock Russet, Belle de Fontenay, BF-15, Bildtstar, Bintje, Blazer, Busset, Blue Congo, Bonnotte, British Queens, Cabritas, Camota, Canela Russet, Cara, Carola, Chelina, Chiloé, Cielo, Clavela Blanca, Désirée, Estima, Fianna, Fingerling, Flava, German Butterball, Golden Wonder, Goldrush, Home Guard, Irish Cobbler, Jersey Royal, Kennebec, Kerr's Pink, Kestrel, Keuka Gold, King Edward, Kipfler, Lady Balfour, Langlade, Linda, Marcy, Marfona, Maris Piper, Marquis, Megachip, Monalisa, Nicola, Pachacoñ a, Pike, Pink Eye, Pink, Fir Apple, Primura, Ratte, Record, Red LaSoda, Red Norland, Red Pontiac, Rooster, Russet Norkotah, Selma, Shepody, Sieglinde, Silverton, Russet, Sirco, Snowden, Spunta, Stobrawa, Superior, Vivaldi, Vitelotte, Yellow Finn, Yukon Gold, blue potato varieties (e.g., Cream of the Crop), Igorota, Solibao, Ganza, Eliane, BelRus, Centennial Russet, Century Russet, Frontier Russet, Hilite Russet, Krantz, Lemhi Russet, Nooksack, Norgold Russet, Norking Russet, Russet Nugget, Allegany, Beacon Chipper, CalWhite, Cascade, Castile, Chipeta, Gemchip, Itasca, Ivory Crisp, Kanona, Katandin, Kennebec Story, La Chipper, Lamoka, Monona, Monticello, Norchip, Norwis, Onaway, Chieftain, La Rouge, NorDonna, Norland, Red La Soda, Red Pontiac, Red Ruby, Sangre, Viking, Ontario, Pike, Sebago, Shepody, Snowden, Superior, Waneta, White Pearl, White Roseand, and all genetically modified varieties. More potato varieties are described in Clough et al., Hort Technology, 2010, 20(1):250-256; Potato Variety Handbook, National Institute of Agricultural Botany, 2000; Chase et al., North American Potato Variety Inventory, Potato Association of America, 1988, each of which is incorporated by reference in its entirety.

Traditional potato growth has been divided into five phases. During the first phase, sprouts emerge from the seed potatoes and root growth begins. During the second, photosynthesis begins as the plant develops leaves and branches. In the third phase stolons develop from lower leaf axils on the stem and grow downwards into the ground and on these stolons new tubers develop as swellings of the stolon. This phase is often (but not always) associated with flowering. Tuber formation halts when soil temperatures reach 80° F. (26.7° C.); hence potatoes are considered a cool-season crop. Tuber bulking occurs during the fourth phase, when the plant begins investing the majority of its resources in its newly formed tubers. At this stage, several factors are critical to yield: optimal soil moisture and temperature, soil nutrient availability and balance, and resistance to pest attacks. The final phase is maturation: The plant canopy dies back, the tuber skins harden, and their sugars convert to starches.

Potato can be used to produce alcoholic beverages, food for human and domestic animals. The potato starch can be used in the food industry as thickeners and binders of soups and sauces, in the textile industry as adhesives, and for the manufacturing of papers and boards. Waste potatoes can be used to produce polylactic acid for plastic products, or used as a base for biodegradable packaging. Potato skins, along with honey, are a folk remedy for burns. Fresh potatoes are baked, boiled, or fried and used in a staggering range of recipes: mashed potatoes, potato pancakes, potato dumplings, twice-baked potatoes, potato soup, potato salad and potatoes au gratin, to name a few. Potatoes can also be used to produce French fries (“chips” in the UK) served in restaurants and fast-food chains worldwide or snack foods such as the potato crisp (“chips” in the US). Dehydrated potato flakes are used in retail mashed potato products, as ingredients in snacks, and even as food aid. Potato flour, another dehydrated product, is used by the food industry to bind meat mixtures and thicken gravies and soups. Potato starch provides higher viscosity than wheat and maize starches, and delivers a more tasty product. It is used as a thickener for sauces and stews, and as a binding agent in cake mixes, dough, biscuits, and ice-cream. In eastern Europe and Scandinavia, crushed potatoes are heated to convert their starch to fermentable sugars that are used in the distillation of alcoholic beverages, such as vodka and akvavit.

### Sweet Potato

The sweet potato (Ipomoea batatas) is a dicotyledonous plant that belongs to the family Convolvulaceae. Its large, starchy, sweet-tasting, tuberous roots are an important root vegetable. The young leaves and shoots are sometimes eaten as greens. Of the approximately 50 genera and more than 1,000 species of Convolvulaceae, I. batatas is the only crop plant of major importance—some others are used locally, but many are actually poisonous. The sweet potato is only distantly related to the potato (Solanum tuberosum). Although the soft, orange sweet potato is often mislabeled a “yam” in parts of North America, the sweet potato is botanically very distinct from a genuine yam, which is native to Africa and Asia and belongs to the monocot family Dioscoreaceae.

### Invertases

Invertase (Inv) (EC 3.2.1.26), a.k.a. beta-fructofuranosidase, is an enzyme that catalyzes the hydrolysis of sucrose, which results in fructose and glucose. Related to invertases are sucrases. Invertases and sucrases hydrolyze sucrose to give the same mixture of glucose and fructose. Invertases cleave the O—C(fructose) bond, whereas the sucrases cleave the O—C(glucose) bond.

Potato invertases are described in Bhaskar et al., Plant Physiology, October 2010, Vol. 154, pp. 939-948, Draffehn et al., BMC Plant Biology, 2010, 10:271, Ye et al., J. Agric. Food Chem. 2010 58:12162-12167, and U.S. Pat. No. 7,094,606, each of which is incorporated herein by reference in its entirety. Additional potato invertases are deposited in the GenBank under accession numbers DQ478950.1, JN661859.1, JN661860.1, AY341425.1, JN661854.1, EU622806.1, L29099.1, JN661857.1, JN661855.1, JN661858.1, JN661856.1, JN661853.1, JN661852.1, EU622807.1, X70368.1, JN661862.1, and JN661861.1. Sequences sharing high homology to potato invertases are deposited in the GenBank under accession numbers HH772321.1, HH772323.1, HH772324.1, HH772322.1, AR928219.1, BD073570.1, I61429.1, I29071.1, I64641.1, E54105.1, E16293.1, E08976.1, E09853.1, E07108.1, HH977806.1, I64644.1, I64642.1, I29074.1, and I29072.1. One skilled in the art would be able to identify and isolate additional potato invertase genes based on the known potato invertase genes.

### Sugar Ends

Sugar ends is an internal tuber disorder primarily observed in processing potatoes and mostly effects long tubers such as ‘Russet Burbank’. It shows up as a post-fry darkening of one end of the French fry, usually on the stem end of the tuber.

Sugar ends is different from cold-induced sweetening, which is a phenomenon of accumulation of reducing sugars in cold-stored potato tubers (Dale and Bradshaw, 2003, Progress in improving processing attributes in potato. Trends Plant Sci 8: 310-312; Bhaskar et al., Suppression of the Vacuolar Invertase Gene Prevents Cold-induced Sweetening in Potato, Plant Physiology, October 2010, 154:939-948). Cold induced sweetening is the tuber quality issue after cold storage of tubers of potato (Solanum tuberosum L.) in many cultivars due to the accumulation of hexose sugars in the process. This is caused by the breakdown of starch to sucrose, which is cleaved to glucose and fructose by vacuolar acid invertase. During processing of affected tubers, the high temperatures involved in baking and frying cause the Maillard reaction between reducing sugars and free amino acids, resulting in the accumulation of acrylamide. However, sugar ends refers to the darkening caused by the carmelization of reducing sugars that accumulate at one end near the region of stolon attachment. Sugar ends are typically associated with plants that have had to endure periods of high air and soil temperatures during tuber initiation and early bulking. Without wishing to be bound by any theory, it is believed that high soil temperatures inhibit the conversion of sugars to starch in the tubers, increasing the concentration of reducing sugars in the affected tissues (Thompson et al. Am. J. Potato Res. 85(5): 375-386 2008). Water deficit at this critical time may also exacerbate sugar ends by interfering with the transport of sugars between tissues. Management options growers have to combat sugar ends include ensuring that moisture stress is minimized during early tuber bulking and creating an environment where the foliage canopy is rapidly attained and preserved over the season. Sugar ends can force farmers to grow potatoes in regions and fields where the potential to grow a high quality crop is maximized. Zebra chip

A new biotic stress of concern to potato growers is Zebra chip caused by the bacterium Candidatus Liberobacter solanacearum. See Secor et al. (Association of ‘Candidatus Liberibacter solanacearum’ with Zebra Chip Disease of Potato Established by Graft and Psyllid Transmission, Electron Microscopy, and PCR, Plant Diseases, 93(6):574-583), and Liefting et al., (‘Candidatus Liberibacter solanacearum’, associated with plants in the family Solanaceae, International Journal of Systematic and Evolutionary Microbiology, 2009, 59(9):2274-2276). Zebra chip (ZC), first discovered in South Texas in 2000, has spread to all major potato production states west of the Mississippi River. It is also a major problem in Guatemala, Honduras, Mexico and New Zealand, causing yield losses and quality issues in tubers that are set on infected plants. Currently, there is no genetic resistance known to the ZC pathogen. Growers can only spray insecticides to thwart the insect vector of the disease, the potato psyllid (Bactericera cockerelli). The ZC pathogen causes infected tubers to exhibit dramatic striped patterns of dark and light discoloration upon chipping and frying. The characteristic striping is evident from heavily infected tubers showing advanced cell death and from lightly infected tubers not having any visible cell death.

Zebra chip infected tubers have elevated levels of phenolic compounds and tyrosine which could account for the rapid browning response of cut tubers (Navarre et al., Amer. J. Potato Res. 86:88-95 2009). Zebra chip-diseased potato tubers are characterized by increased levels of host phenolics, amino acids, and defense-related proteins. (Wallis et al. Physiological and Molecular Plant Pathology 78 (2012) 66-72). Because silencing of polyphenol oxidase (Ppo) has been linked to reduced symptom expression in tubers before (Rommens et al. J. Agric. Food Chem. 2006, 55, 9882-9887), it would be assumed that Ppo silencing could reduce the symptoms of ZC in infected tubers. However, because there is also the assumption that Ppo silencing could be associated with heightened disease susceptibility (Thipyapong et al. Planta 2004, 220, 105-117), Ppo silencing may only worsen the symptoms and severity of ZC. The present invention confirms a heightened Ppo response in ZC-infected tubers but do not show the ability to reduce carmelization color in fried potatoes infected with ZC. Moreover, it was not possible to show a reduced symptom development in the Ppo silenced versus non-Ppo silenced lines. Each of reference mentioned above is incorporated herein by reference in its entirety.

Candidatus Liberibacter is a genus of gram-negative bacteria in the Rhizobiaceae family. The term Candidatus is used to indicate that it has not proved possible to maintain this bacterium in culture. Detection of the liberibacters is based on PCR amplification of their 16S rRNA gene with specific primers. Members of the genus are plant pathogens mostly transmitted by psyllids. The genus was originally spelled Liberobacter. Non-limiting species of Candidatus Liberibacter include Liberibacter africanus, Liberibacter americanus, Liberibacter asiaticus, Liberibacter europaeus, Liberibacter psyllaurous, and Liberibacter solanacearum.

The complete genome sequence of ‘Candidatus Liberibacter solanacearum’ has been disclosed (Lin et al., The Complete Genome Sequence of ‘Candidatus Liberibacter solanacearum’, the Bacterium Associated with Potato Zebra Chip Disease, PLOS One, 201, 6(4):e19135). Preliminary transmission trials strongly suggested that B. cokerelli is a vector of ‘Ca. L. solanacearum’. It has been demonstrated that the psyllid can acquire the bacterium but transmission needs to be confirmed. In addition, many other aspects of the disease epidemiology remain to be studied (e.g. transmission through seeds or grafts). Over long distances, trade of infected plants and psyllids can spread the bacterium. ‘Ca. L. solanacearum’ has been found in association with other psyllid species, B. trigonica and T. apicalis, and also in mixed infections with other pathogens (e.g. Aster yellows phytoplasma, Spiroplasma citri).

The existence or absence of ‘Candidatus Liberibacter solanacearum’ can be detected by any method known to one skilled in the art, for example, by observing the Zebra chip symptoms in the potato tubers, or by methods based on nucleotides hybridization, such as conventional or Real-time PCR (Crosslin et al., “Detection of ‘Candidatus Liberibacter solanacearum’ in the Potato Psyllid, Bactericera cockerelli (Sulc), by Conventional and Real-Time PCR, Southwestern Entomologist, 36(2):125-135, 2011). Other methods include, but are not limited to immunological detection tests selected from the group consisting of precipitation and agglutination tests, immunogold labeling, immunosorbent electron microscopy, ELISA (e.g., Lateral Flow test, or DAS-ELISA), Western blot, RIA, and/or dot blot test, and combination thereof.

### Methods

The present invention provides methods of producing potato tubers with lower incidence of sugar ends in potato products such as French fries or chips. The present invention also provides methods for making potato products that are mildly infected with the zebra chip pathogen but with less severe symptoms, e.g., having less off-color development after being fried, despite the presence of low titers of the pathogen.

The incidence of sugar ends in potato products can be evaluated by methods known to one skilled in the art, such as the one described in Example 1 below. In some embodiments, the color of the potato products made from potato tubers to be tested is used as an indicator of sugar ends and measured against potato products made from a control potato tuber with the help of a color chart, such as the USDA Munsell Color Chart for potato products. Suitable control potato tubers can be any corresponding potato varieties having un-disrupted invertase while the control potato tubers have been grown, harvested, and treated under the same conditions as the potato tubers to be tested. In some embodiments, the percentage of potato products made from potato tubers of the present invention having sugar ends phenotype is significantly lower than that of a control potato tuber. For example, the percentage of potato products made from potato tubers of the present invention having sugar ends phenotype is about 0%, 1%, 2%, 3%, 4%, 5%, 6%, 7%, 8%, 9%, 10%, 11%, 12%, 13%, 1%, 15%, 16%, 17%, 18%, 19% 20%, 21%, 22%, 23%, 24%, 25%, 26%, 27%, 28%, 29%, or 30%.

The symptoms of Zebra Chip (ZC) pathogen in potato products can be evaluated by methods known to one skilled in the art, such as the one described in Example 2 below. In some embodiments, a visual estimation of ZC severity (i.e., necrotic flecking of the tuber flesh) can be made on the tubers after the plants are treated with the pathogen. During the assessment of symptom severity, tuber samples were taken for PCR verification for the presence or absence of Liberibacter. The presence of ZC is correlated with increasingly darker chips the longer the plants were exposed to the Liberibacter-positive pysllids. In some embodiments, for the fried products, the products can be fried in oil for about 1, 2, 3, 4, 5 or more minutes at about 300 F, 350 F, 400 F or 450 F to achieve about 1%, 2%, 3%, %, 5% final moisture in the products before comparison. In some embodiments, the color development of the products is examined by visual observation and reflected in the Agtron readings. Higher Agtron readings are correlated with lighter color. The products made from potato tubers with disrupted invertase gene have lighter color compared to the products made from a control potato tuber, indicating less severe symptoms.

In some embodiments, the methods comprise disrupting an invertase gene/enzyme activity in said potato plant. In some embodiments, the invertase is a vacuolar invertase. In some embodiments, the invertase gene/enzyme activity is disrupted at least in the potato tuber. In some embodiments, the invertase gene/enzyme activity is only disrupted in the potato tuber. As used herein, the term “disrupted”, “disrupting” or “disruption” refers to that the vacuolar invertase enzyme activity in a potato plant is modified in a way so that it is lowered, reduced or even completely abolished compared to the invertase enzyme activity in a control plant.

Methods of disrupting the activity of an enzyme have been known to one skilled in the art. These methods include, but are not limited to, mutagenesis (e.g., chemical mutagenesis, radiation mutagenesis, transposon mutagenesis, insertional mutagenesis, signature tagged mutagenesis, site-directed mutagenesis, and natural mutagenesis), knock-outs/knock-ins, antisense and RNA interference. Various types of mutagenesis can be used to produce and isolate potato plants with disrupted vacuolar invertase enzyme activity. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present invention. In one embodiment, mutagenesis can be guided by known information of the naturally occurring molecule or altered or mutated naturally occurring molecule, e.g., sequence, sequence comparisons, physical properties, crystal structure or the like. For more information of mutagenesis in plants, such as agents, protocols, see Acquaah et al. (Principles of plant genetics and breeding, Wiley-Blackwell, 2007, ISBN 1405136464, 9781405136464, which is herein incorporated by reference in its entity).

In some embodiments, the methods comprise disrupting the activity of the endogenous invertase gene in a potato plant by using one or more inhibitory nucleotide sequences, such as nucleotide sequences for RNA interference, antisense oligonucleotides, microRNA, and/or steric-blocking oligonucleotides (See Kole et al., RNA therapeutics: beyond RNA interference and antisense oligonucleotides, Drug Discovery, 2012, 11:125-140; Ossowski et al., Gene silencing in plants using artificial microRNAs and other small RNAs, The Plant Journal, 2008, 53(4):674-690; Wang et al., Application of gene silencing in plants, Current Opinion in Plant Biology, 2002, 5(2):146-150; Vaucheret et al., Post-transcriptional gene silencing in plants, Journal of Cell Science, 2001, 114:3083-3091; Stam et al., Review Article: The Silence of Genes in Transgenic Plants, annals of Botany, 79(1):3-12; Highly Specific Gene Silencing by Artificial MicroRNAs in Arabidopsis, The Plant Cell, 2006, 18(5):1121-1133; David Allis et al., Epigenetics, CSHL Press, 2007, ISBN 0879697245, 978087969724; Sohail et al., Gene silencing by RNA interference: technology and application, CRC Press, 2005, ISBN 0849321417, 9780849321412; Engelke et al., RAN Interference, Academic Press, 2005, ISBN 0121827976, 9780121827977; and Doran et al., RNA Interference: Methods for Plants and Animals, CABI, 2009, ISBN 1845934105, 9781845934101, each of which is incorporated herein by reference in its entirety for all purposes).

The inhibitory nucleotide sequences can be operably linked to a plant promoter, such as a constitutive promoter, a non-constitutive promoter, an inducible promoter, a tissue specific promoter, or a cell-type specific promoters.

RNA interference (RNAi) is the process of sequence-specific, post-transcriptional gene silencing or transcriptional gene silencing in animals and plants, initiated by double-stranded RNA (dsRNA) that is homologous in sequence to the silenced gene. The preferred RNA effector molecules useful in this invention must be sufficiently distinct in sequence from any host polynucleotide sequences for which function is intended to be undisturbed after any of the methods of this invention are performed. Computer algorithms may be used to define the essential lack of homology between the RNA molecule polynucleotide sequence and host, essential, normal sequences.

The term “dsRNA” or “dsRNA molecule” or “double-strand RNA effector molecule” refers to an at least partially double-strand ribonucleic acid molecule containing a region of at least about 19 or more nucleotides that are in a double-strand conformation. The double-stranded RNA effector molecule may be a duplex double-stranded RNA formed from two separate RNA strands or it may be a single RNA strand with regions of self-complementarity capable of assuming an at least partially double-stranded hairpin conformation (i.e., a hairpin dsRNA or stem-loop dsRNA). In various embodiments, the dsRNA consists entirely of ribonucleotides or consists of a mixture of ribonucleotides and deoxynucleotides, such as RNA/DNA hybrids. The dsRNA may be a single molecule with regions of self-complementarity such that nucleotides in one segment of the molecule base pair with nucleotides in another segment of the molecule. In one aspect, the regions of self-complementarity are linked by a region of at least about 3-4 nucleotides, or about 5, 6, 7, 9 to 15 nucleotides or more, which lacks complementarity to another part of the molecule and thus remains single-stranded (i.e., the “loop region”). Such a molecule will assume a partially double-stranded stem-loop structure, optionally, with short single stranded 5′ and/or 3′ ends. In one aspect the regions of self-complementarity of the hairpin dsRNA or the double-stranded region of a duplex dsRNA will comprise an Effector Sequence and an Effector Complement (e.g., linked by a single-stranded loop region in a hairpin dsRNA). The Effector Sequence or Effector Strand is that strand of the double-stranded region or duplex which is incorporated in or associates with RISC. In one aspect the double-stranded RNA effector molecule will comprise an at least 19 contiguous nucleotide effector sequence, preferably 19 to 29, 19 to 27, or 19 to 21 or more nucleotides, which is a reverse complement to the RNA of the invertase gene, or an opposite strand replication intermediate.

In one embodiment, said double-stranded RNA effector molecules are provided by providing to a potato plant, plant tissue, or plant cell an expression construct comprising one or more double-stranded RNA effector molecules. In one embodiment, the expression construct comprises a double-strand RNA derived from the invertase gene in potato.

In some embodiments, the dsRNA effector molecule of the invention is a “hairpin dsRNA”, a “dsRNA hairpin”, “short-hairpin RNA” or “shRNA”, i.e., an RNA molecule of less than approximately 400 to 500 nucleotides (nt), or less than 100 to 200 nt, in which at least one stretch of at least 15 to 100 nucleotides (e.g., 17 to 50 nt, 19 to 29 nt) is based paired with a complementary sequence located on the same RNA molecule (single RNA strand), and where said sequence and complementary sequence are separated by an unpaired region of at least about 4 to 7 nucleotides (or about 9 to about 15 nt, about 15 to about 100 nt, about 100 to about 1000 nt) which forms a single-stranded loop above the stem structure created by the two regions of base complementarity. The shRNA molecules comprise at least one stem-loop structure comprising a double-stranded stem region of about 17 to about 500 bp; about 17 to about 50 bp; about 40 to about 100 bp; about 18 to about 40 bp; or from about 19 to about 29 bp; homologous and complementary to a target sequence to be inhibited; and an unpaired loop region of at least about 4 to 7 nucleotides, or about 9 to about 15 nucleotides, about 15 to about 100 nt, about 250-500 bp, about 100 to about 1000 nt, which forms a single-stranded loop above the stem structure created by the two regions of base complementarity. It will be recognized, however, that it is not strictly necessary to include a “loop region” or “loop sequence” because an RNA molecule comprising a sequence followed immediately by its reverse complement will tend to assume a stem-loop conformation even when not separated by an irrelevant “stuffer” sequence.

The expression constructs of the present invention comprising DNA sequence which can be transcribed into one or more double-stranded RNA effector molecules can be transformed into a potato plant, wherein the transformed plant has disrupted invertase activity. The target sequence to be inhibited by the dsRNA effector molecule include, but are not limited to, coding region.

In some embodiments, the RNAi constructs of the present invention comprise one or more inverted repeats. The inverted repeats can be transcribed into interference RNA molecules in the potato plants. In some embodiments, the transcribed interference RNA molecules can target the promoter region, the coding region, the intron, the 5′ UTR region, and/or the 3′ UTR region of the invertase gene in the potato.

In some embodiments, the inverted repeats comprise a sense strand and an anti-sense strand. In some embodiments, the sense stand and the anti-sense stand are perfectly complementary to each other. In some embodiments, the sense stand and the anti-sense stand are not perfectly complementary to each other for the full length, but are at least complementary partially. In some embodiments, the sense stand shares about 70%, about 80%, about 90%, about 95%, about 99% or more homology to the invertase gene in the potato. In some embodiments, the sense stand comprises a fragment corresponding to +53 to +733 of the invertase gene (which can be amplified by primers SEQ ID NO: 1 and SEQ ID NO: 19). In some embodiments, the anti-sense strand comprises a fragment corresponding to +552 to +49 of the invertase gene (which can be amplified by primers SEQ ID NO: 2 and SEQ ID NO: 20). In some embodiments, the sense strand and/or the anti-sense strand comprises a fragment corresponding to 673-1168, 1310-1818, or 1845-2351 of the invertase gene.

In some embodiments, the invertase activity is at least interrupted in potato tubers. In some embodiments, the invertase activity is only or mainly interrupted in potato tubers. To achieve tuber-specific interruption, the invertase silencing polynucleotides of the present invention can be driven by one or more tuber-specific promoter. Non-limiting examples of tuber-specific promoters include those described in Ye et al., 2010 (e.g., the promoter associated with the ADP glucose pyrophosphorylase (AGP) gene, such as SEQ ID NO: 6, or functional variants, fragments thereof), Twell et al., (Plant Molecular Biology, 9:365-375 (1987) S. Rosahl et al., (“The 5′ Flanking DNA of a patatin gene directs tuber specific expression of a chimaeric gene potato”, “Organ-Specific Gene Expression in Potato: Isolation and Characterization of Tuber-Specific cDNA Sequences”, Molecular Gen Genet, (1986) 202: pp. 368-373), and U.S. Pat. No. 5,436,393 (e.g., B33 promoter sequence of a patatin gene derived from Solanum tuberosum, or functional variants, fragments thereof), U.S. Pat. No. 6,184,443(e.g., promoter sequence of the potato α-amylase gene, or functional variants, fragments thereof), each of which is incorporated herein by reference in its entirety.

In some embodiments, the methods comprise disrupting an invertase activity by screening potato plants having naturally mutated invertase gene. Alternatively, potato plants can be mutagenized by methods known to one skilled in the art, and potato plants with mutated invertase gene can be identified and isolated.

In some embodiments, the potato plants in which the invertase is disrupted have one or more agriculturally important traits. As used herein, “agronomically important traits” include any phenotype in a plant or plant part that is useful or advantageous for human use. Examples of agronomically important traits include but are not limited to those that result in increased biomass production, production of specific biofuels, increased food production, improved food quality, increased seed oil content, etc. Additional examples of agronomically important traits includes pest resistance, vigor, development time (time to harvest), enhanced nutrient content, novel growth patterns, flavors or colors, salt, heat, drought and cold tolerance, and the like. In some embodiments, the agriculturally important traits of a potato plant include, but are not limited to traits related to Adaptability, After cooking blackening, Berries, Cooking type, Cooked texture, Crisp suitability, Dormancy period, Drought resistance, Dry matter content, Early harvest yield potential, Enzymic browning, Field immunity to wart races, Flower colour, Flower frequency, Foliage cover, French fry suitability, Frost resistance, Frying colour, Growth cracking, Growth habit, Hollow heart tendency, Internal rust spot, Light sprout colour, Maturity, Pollen fertility, Presence of late blight R gene, Primary tuber flesh colour, Protein content, Rate of bulking, Resistance to aphids, Resistance to bacterial soft rot (Erwinia spp.), Resistance to bacterial wilt (Ralstonia solanacearum), Resistance to blackleg (Erwinia spp.), Resistance to common scab (Streptomyces scabies), Resistance to dry rot (Fusarium coeruleum), Resistance to dry rot (Fusarium spp.), Resistance to dry rot (Fusarium sulphureum), Resistance to early blight (Alternaria solani), Resistance to external damage, Resistance to fusarium wilt (Fusarium oxysporum), Resistance to gangrene (Phoma foveata), Resistance to Globodera pallid, Resistance to Globodera rostochiensis, Resistance to internal bruising, Resistance to late blight on foliage, Resistance to late blight on tubers, Resistance to potato leaf roll virus, Resistance to potato mop top virus, Resistance to potato virus (e.g., A, B, C, MS, X, Y, YN), Resistance to powdery scab (Spongospora subterranea), Resistance to ring rot (Clavibacter michiganensis ssp. sepedonicus), Resistance to slugs, Resistance to stem canker (Rhizoctonia solani), Resistance to tobacco rattle virus, Resistance to tuber moth, Sample status, Secondary growth, Secondary tuber flesh colour, Starch content, Stolon attachment, Stolon length, Storage ability, Susceptibility to wart races, Taste, Test conditions, Tuber eye colour, Tuber eye depth, Tuber glycoalkaloid, Tuber greening before harvest, Tuber shape, Tuber shape uniformity, Tuber size, Tuber skin colour, Tuber skin, texture, Tubers per plant, Wart (Synchytrium endobioticum), and Yield potential.

The present invention also provides methods for breeding potato plants which produce potato tubers having lower incidence of sugar ends, and/or potato tubers having less off-color development when mildly infected with the zebra chip pathogen. In some embodiments, the methods comprise (i) crossing any one of the plants of the present invention comprising a disrupted invertase gene as a donor to a recipient plant line to create a F1 population; (ii) evaluating the sugar ends and/or Zebra Chip phenotypes in the offsprings derived from said F1 population; and (iii) selecting offsprings that produce potato tubers having lower incidence of sugar ends, and/or potato tubers having less off-color development when mildly infected with the zebra chip pathogen. In some embodiments, the recipient plant is an elite line having one or more certain agronomically important traits.

### Plant Transformation

The most common method for the introduction of new genetic material into a plant genome involves the use of living cells of the bacterial pathogen Agrobacterium tumefaciens to literally inject a piece of DNA, called transfer or T-DNA, into individual plant cells (usually following wounding of the tissue) where it is targeted to the plant nucleus for chromosomal integration. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. No. 5,693,512, U.S. Pat. No. 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with P-DNA are also well known to those skilled in the art and can have applicability in the present invention. See, for example, U.S. Pat. No. 7,250,554, which is incorporated herein by reference in its entirety.

Non-limiting examples of potato transformation methods are described in U.S. Pat. Nos. 7,534,934, 8,273,949, 7,855,319, 7,619,138, 7,947,868, 8,193,412, 7,880,057, 8,252,974, 7,250,554, 8,143,477, 8,137,961, 7,601,536, 7,923,600, 7,449,335, 7,928,292, 7,713,735, 8,158,414, 7,598,430, 5,185,253, Beaujean et al., (Agrobacterium-mediated transformation of three economically important potato cultivars using slice intermodal explants: an efficient protocol of transformation, Journal of Experimental Botan, 49(326):1589-1595), Chakravarty et al., (Rapid regeneration of stable transformants in cultures of potato by improving factors influencing Agrobacterium-mediated transformation, Advances in Bioscience and Biotechnology, 2010, 1:409-416), Barrell et al., (Alternative selectable markers for potato transformation using minimal T-DNA vectors, Plant Cell, Tissue and Organ Culture, Volume 70, Number 1 (2002), 61-68), Andersson et al., (A novel selection system for potato transformation using a mutated AHAS gene, Plant Cell Rep., 2003, 22(4):261-267), Valkov et al., (High efficiency plastid transformation in potato and regulation of transgene expression in leaves and tubers by alternative 50 and 30 regulatory sequences, Transgenic Res (2011) 20:137-151), and Tavazza et al (Genetic transformation of potato (Solanum tuberosum): An efficient method to obtain transgenic plants, Plant Science, Volume 59, Issue 2, 1989, Pages 175-181), each of which is incorporated herein by reference in its entirety.

### Breeding Methods

General breeding methods for potato is described in, but not limited to Hybridization of crop plants (American Society of Agronomy and Crop Science Society of America, 1980, Chapter 34), Bradshaw et al., (Genetic Resources and Progress in Their Utilization in Potato Breeding, Potato Research, 2006, 49:49-65), Barone (Molecular Marker-assisted Selection for Potato Breeding, Amer. J. of Potato Res. 2004, 81:111-117), Douches et al., (Assessment of Potato Breeding Progress in the USA over the Last Century, Crop Science, 36(6):1544-1552), Advances in Potato chemistry and Technology (Academic Press, 2009, ISBN 0123743494, 9780123743497, Chapter 8, Potato Breeding Strategy, Bradshaaw), and Janick et al. (Potato Breeding via Ploidy Manipulations, Plant Breeding Reviews, 2010). Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety.

Classic breeding methods can be included in the present invention to introduce one or more recombinant expression cassettes of the present invention into other plant varieties, or other close-related species that are compatible to be crossed with the transgenic plant of the present invention.

Open-Pollinated Populations.

The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes to flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection.

In mass selection, desirable individual plants are chosen, harvested, and the seed composited without progeny testing to produce the following generation. Since selection is based on the maternal parent only, and there is no control over pollination, mass selection amounts to a form of random mating with selection. As stated herein, the purpose of mass selection is to increase the proportion of superior genotypes in the population.

Synthetics.

A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

Pedigreed Varieties.

A pedigreed variety is a superior genotype developed from selection of individual plants out of a segregating population followed by propagation and seed increase of self pollinated offspring and careful testing of the genotype over several generations. This is an open pollinated method that works well with naturally self pollinating species. This method can be used in combination with mass selection in variety development. Variations in pedigree and mass selection in combination are the most common methods for generating varieties in self pollinated crops.

Hybrids.

A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Invertase Silencing to Minimize the Incidence of Sugar Ends in Field-Stressed Tubers

Sense and antisense fragments of the cDNA of the Inv gene (Genbank AccessionDQ478950) were amplified from a tuber poly(A)+mRNA-derived library of the potato variety ‘Ranger’ Russet using the two primer pairs (SEQ ID NO: 1 and SEQ ID NO: 19; SEQ ID NO: 2 and SEQ ID NO: 20). The amplified fragments corresponded to positions +53 to +733 (sense) and +552 to +49 (antisense), respectively, of the Inv gene. Any fragment down to 21-23 base pairs of the invertase cDNA could be used to silence the Inv gene (SEQ ID NO: 5). The cloned fragments were positioned as inverted repeats (SEQ ID NOs: 3 and 4) between regulatory elements from the potato variety ‘Ranger’ Russet: the 2.2 kb tuber-specific promoter of the ADP glucose pyrophosphorylase (Agp) gene (Accession HM363752, SEQ ID NO: 6) and the 0.3 kb terminator of the ubiquitin-3 gene (AccessionGP755544, SEQ ID NO: 7). Insertion of the resulting silencing cassette into a pSIM401-derived T-DNA region also carrying an expression cassette for the selectable marker neomycin phosphotransferase (npt) gene (Rommens et al., Plant Physiol. 139: 1338-1349 2005) yielded vector pSIM1632.

Agrobacterium harboring the pSIM1632 Inv silencing vector was grown overnight at 28° C. in LB medium (20 g/L LB Broth, Sigma) containing antibiotics to select for bacteria and vector. Ten-fold dilutions of the overnight cultures were grown 5-6 hours to log phase and precipitated at 3000 rpm. The pellet was washed in M404 liquid medium (PhytoTechnology, Shawnee, Kans.) supplemented with 3% sucrose and resuspended in the same liquid medium to obtain a cell density of OD₆₀₀ of 0.2.

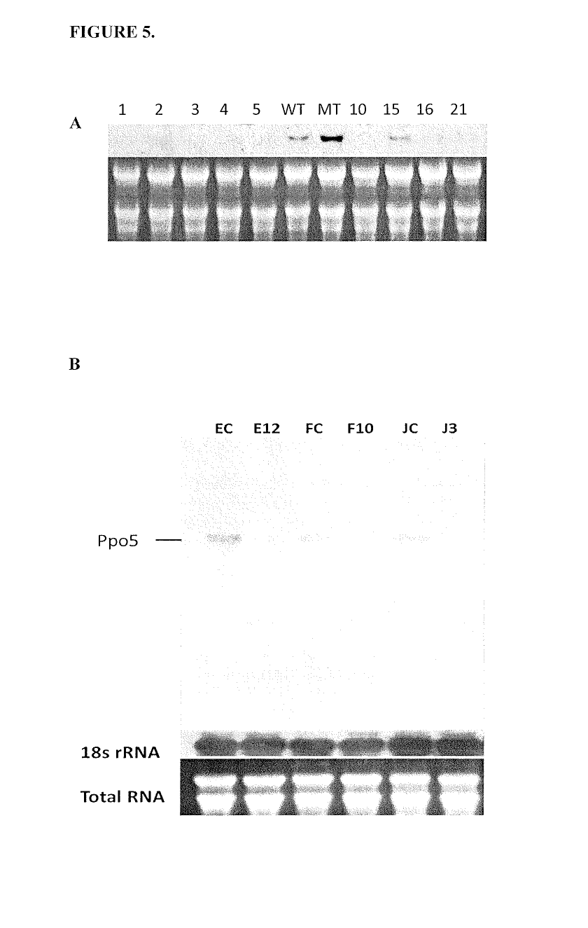

Stock plants for explant material was maintained in magenta boxes with 40 ml of half-strength M516 medium (PhytoTechnology, Shawnee, Kans.) containing 3% sucrose and 2 g/L gelrite, pH 5.7. Potato internodal segments of 4-6 mm were cut from 4-week old plants, infected with Agrobacterium and transferred to M404 medium supplemented with 3% sucrose and 6 g/L agar, pH 5.7. After 2 days co-cultivation, explants are placed on callus induction medium which is M404 medium plus 3% sucrose, 2.5 mg/L zeatin riboside, 0.1 mg/L NAA, 6 g/L agar, pH 5.7 and 150 mg/L timentin to eliminate Agrobacterium and 100 mg/L kanamycin as selection agent. After a month on callus induction medium, explants are moved to shoot induction medium (M404 medium plus 3% sucrose, 2.5 mg/L zeatin riboside, 0.3 mg/L GA₃, 6 g/L agar, pH 5.7, 150 mg/L timentin and 100 mg/L kanamycin) until shoots are obtained. Shoots are rooted on M404 medium plus 3% sucrose, gelling agent and 100 mg/L kanamycin. Shoots rooting in presence of kanamycin are screened via PCR for the presence of the transgene. Northern analyses confirm the silencing of the Inv gene in the lines selected for the ZC experiment (FIG. 5A). Lines silenced for the gene of interest are propagated in vitro and grown in the greenhouse for seed production.

Field trials using untransformed controls, empty vector controls and invertase-silenced lines were conducted in Year 1 and Year 2 at University of Idaho Parma Research and Extension Center in Parma, Id. Applications of macro and micronutrients followed management recommendations suggested by the University of Idaho. Plots were sprinkled irrigated using a solid set system with moisture maintained above 65% throughout the growing season. In Year 1, each control and transgenic line was represented by 1 plot of 5 hills. In Year 2, each control and transgenic line was represented by 5 plots of 20 hills. In both years, in-row spacing was 10 inches with 36 inches between rows. Tubers were harvested 130-140 days from planting and stored at 55° C. until frying (about 2 weeks).

In Year 1, a fry sample consisted of a minimum of twelve pounds of tubers taken from a pooled sample of the 5 hills. In Year 2, 20 tubers from a pooled conglomeration each replicate of 20 hills were used and all 5 replicates were measured. The Year 2 average number of tubers per line was 5×20 or 100 tubers. All tubers were cut lengthwise on a ⅜-inch×⅜-inch grid fry knife and the four center strips were fried at 375 degrees F. for 3 minutes. Fried strips are laid on a white tray and compared to the USDA Munsell Color Chart for French Fried Potatoes. A SE fry has an end ¼ inch long or longer on the darkest two sides of the strip, for the full width of the strip, testing number 3 or darker when compared to the USDA Munsell Color Chart.

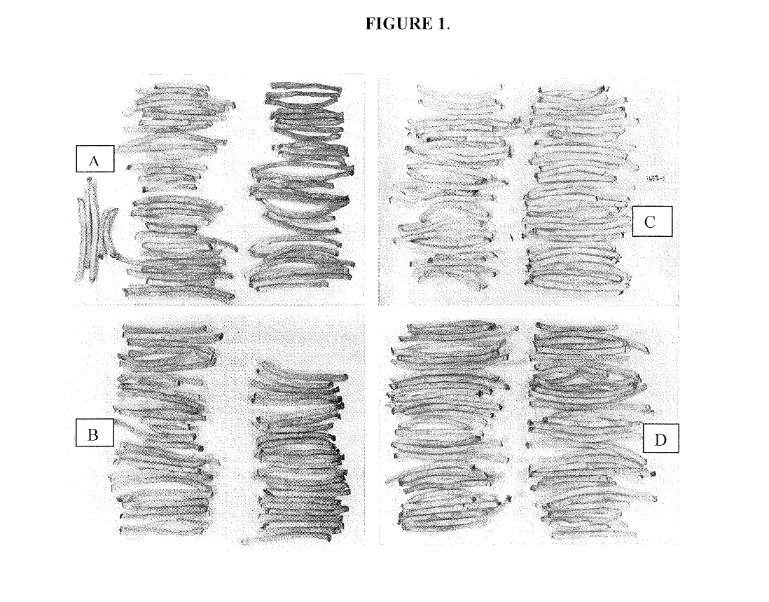

As shown in Table 1, conditions suitable to the induction of sugar ends were present in the Parma, Id. field in both years. In Year 1, a small sample size due to limited seed supply revealed trends toward all lines having reduced sugar ends. Although nearly half of the center strip fries of untransformed control (Ranger control) and the empty vector control show sugar ends, invertase-silenced lines all show dramatic reductions. This fact is also apparent from the illustration in FIG. 1 which shows all of the center strip fries for each sample. Strikingly few of the invertase-silenced lines showed any fries with sugar ends as illustrated in FIG. 1. Lines 1 and 4 were marked by no sugar ends in the samples fried. Other invertase lines showed less than 14% of fries with sugar ends versus 42% of control fries showing sugar ends upon frying. The same pattern where the invertase silenced lines showed considerable reductions in sugar ends was observed in Year 2 when more replication was possible. Line 1632-1 was excellent in both years with only an average of 4±2.3 (± standard deviation) French fries showing any degree of sugar ends. Two replicates had no strips with sugar ends. Other lines showed less reduction in Year 2, demonstrating the importance of larger sample size when studying sugar ends.

TABLE 1
The frequency of center cut French fries with sugar ends (SE) from
invertase-silenced Russet Ranger (1632-x), empty vector control and
untransformed (Ranger control) tubers. A SE fry has an end ¼
inch long or longer on the darkest two sides of the strip (the length
of darker zone used in the fry industry for measurement), for the full
width of the strip, testing number 3 or darker when compared to the
USDA Munsell Color Chart for French Fried Potatoes.

| | Year 1* | | Year 2** |
|---|---|---|---|
| | No. fries | No. fries | Mean % French fries/ |
| Line ID | scored | with SE | rep with SE^{§} |
| 1632-1 | 60 | 0 | 4.0 ± 2.3 |
| 1632-3 | 60 | 4 | 28.4 ± 2.4 |
| 1632-4 | 60 | 0 | 36.1 ± 2.8 |
| 1632-5 | 60 | 8 | 20.1 ± 7.9 |
| 1632-21 | 60 | 4 | 30.5 ± 5.0 |
| empty vector | 51 | 24 | 38.6 ± 8.0 |
| Ranger control | 60 | 25 | 50.0 ± 7.3 |

*No replication due to limited amount of seed.
**Each line and control replicated 5 times.
^{§}Average number of French fries with sugar ends ± std deviation

### Example 2

### Invertase Silencing to Minimize the Severity of Zebra Chip-Induced Darkening of Fried Potato Products Like Chips and French Fries

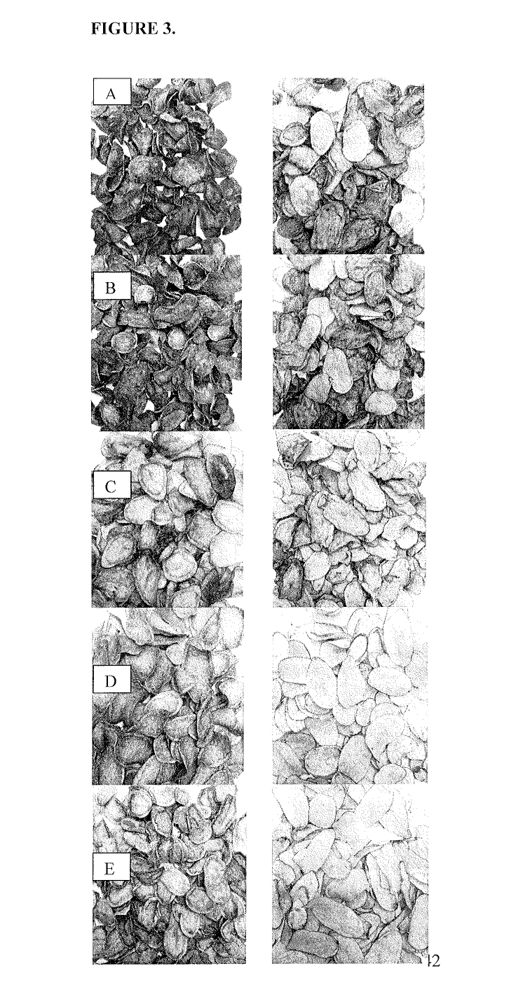

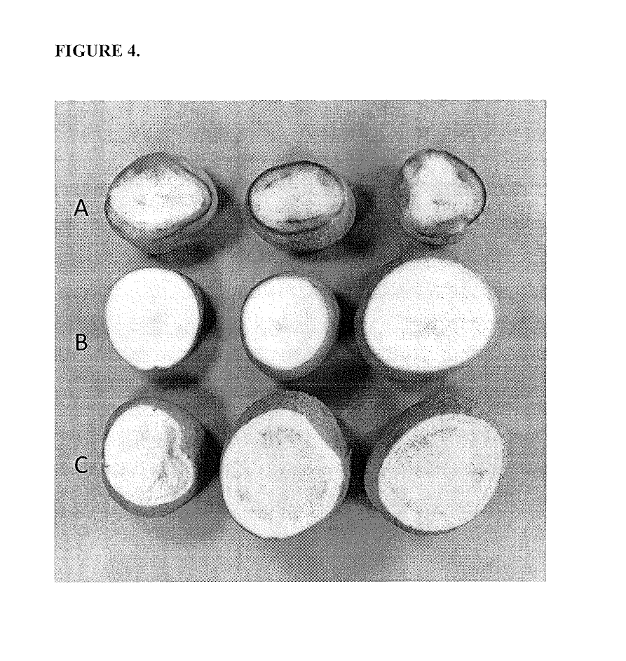

The generation of the invertase-silenced lines used in the Zebra chip (ZC) experiments was described above. The same lines showing reduced frequency of sugar ends were tested for ability to minimize the color generation in chips infected by the causal agent of Zebra chip. A field trial using greenhouse-grown seed for the untransformed controls, empty vector controls and invertase-silenced lines was conducted at Texas A&M University Bushland Research and Extension Center in Bushland, Tex. Seed was planted April 11. Four plants per treatment were planted in a block and covered by a tent after emergence. The tents served to keep unwanted fauna from the plants and hold infected psyllids—the vector of the ZC causal organism—on the plants. Four plants of each line contained within the tents were infected with 30 psyllids carrying Liberibacter at 35, 28, 21, 14, and 7 days before harvest. In this way, tubers were generated from each line and controls that were progressively more or less infected with Zebra chip. Plants infected at 35 days prior to harvest would likely be systemically infected and show very strong symptoms of ZC (FIG. 3B and FIG. 4) with the resulting chips frying up very dark. Plants infected 21 days prior to harvest is expected to show only very mild infection symptoms in the tubers (FIG. 3A) and would likely fry up with a moderate amount of darkening. A plant infected only 7 days prior to harvest is expected to show little or no signs of infection and would likely have tubers that would fry up with little or no darkening.

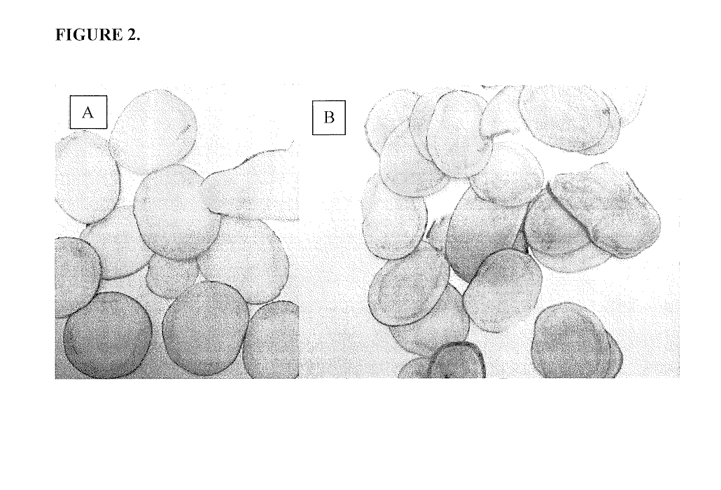

At harvest, tubers from each line and treatment were analyzed for ZC symptoms. A visual estimation of ZC severity (i.e., necrotic flecking of the tuber flesh) was made on eight tubers from each treatment. The stolon end was cut and a 0 to 3 rating was given to the tuber for symptoms with a 3 showing the greatest amount of tuber necrosis and a 0 showing no necrosis. Table 2 summarizes the disease severity scores for each line at each infection time. As expected, control tubers showed signs of severe infection at the 35 and 28 days before harvest (dbh) with obvious spots and streaks of necrotic tissue throughout the tuber flesh (see FIG. 2A). Tubers infected 21 dbh may occasionally show signs of light necrotic flecking in the cortex of the tuber as shown in FIG. 2B. Progressively fewer signs of infection marked by little or no necrosis were apparent at days closer to harvest. The invertase-silenced lines scored no better than the untransformed ‘Ranger’ Russet control, showing that fresh symptoms cannot be alleviated by the silencing of invertase. During the assessment of symptom severity, tuber samples were taken for PCR verification for the presence or absence of Liberibacter.

TABLE 2
Average disease severity ratings of ZC-infected and uninfected tubers. Polyphenol oxidase silenced
lines and invertase silenced lines are indicated with the appropriate untransformed controls. Eight
tubers from each treatment were cut at the stolon end and rated on a scale between 0 and 3, with
a 3 corresponding to the greatest amount of tuber necrosis and a 0 showing no necrosis. Value represented
is an average of all eight tubers. DBH = days before harvest. J3, E12 and F10 are Ppo-silenced
lines in the ‘Atlantic’ (Atl), Russet Burbank (RB) and ‘Ranger’ Russet (RR) backgrounds,
respectively. All 1632 lines are silenced for Inv in the Russet ‘Ranger’ background.

| | J3 | Atl | E12 | RB | F10 | RR | 1632-1 | 1632-3 | 1632-4 | 1632-5 | 1632-21 |
|---|---|---|---|---|---|---|---|---|---|---|---|
| 35 days dbh | 1.88 | 1.00 | 2.69 | 1.88 | 1.75 | 2.00 | 1.94 | 2.56 | 1.63 | 1.56 | 1.63 |
| 28 days dbh | 2.13 | 1.06 | 1.75 | 1.31 | 1.75 | 1.42 | 1.25 | 2.19 | 2.50 | 0.75 | 2.13 |
| 21 days dbh | 0.0 | 0.50 | 0.08 | 0.0 | 0.25 | 0.38 | 0.13 | 0.31 | 0.81 | 0.25 | 0.69 |
| 14 days dbh | 0.0 | 0.19 | 0.0 | 0.0 | 0.56 | 0.31 | 0.13 | 0.25 | 0.06 | 0.31 | 0.13 |
| 7 days dbh | 0.0 | 0.0 | 0.0 | 0.19 | 0.25 | 0.13 | 0.13 | 0.13 | 0.14 | 0.19 | 0.31 |
| No infection | 0.0 | 0.06 | 0.0 | 0.58 | 0.06 | 0.00 | 0.06 | 0.06 | 0.13 | 0.25 | 0.06 |

Chipping of 6-8 control tubers per infection day was performed to see the influence of ZC infection on the color of finished chips. A one pound sample of slices was fried in oil for 3 minutes at 350 F in order to achieve 2% final moisture in the chip. As seen in FIG. 3, the presence of ZC is correlated with increasingly darker chips the longer the plants were exposed to the Liberibacter-positive pysllids. Chip color as measured by Agtron readings becomes less dark in ‘Ranger’ control chips as ZC pressure diminishes at dates closer to harvest (Table 3).

Chipping of 6-8 invertase silenced tubers per infection day was performed to see the influence of invertase silencing on the manifestation of ZC-influence color development in fried chips. As reflected in the Agtron readings and from visible examination of the chip color, the invertase silencing resulted in the lower chip color at every infection time point. Even severely infected tubers were lighter compared to the ‘Ranger’ control tubers; although the chips from 35 and 28 dbh are still unmarketable. Chips from lightly infected tubers (≦21 dbh) would likely all be marketable according the outcome of this experiment.

TABLE 3
Agtron readings for of chips prepared from ZC-infected and uninfected
tubers with and without invertase silencing. Each value is an
average of 3 readings on the same sample. Certain data points
are missing due to crop failure. Higher numbers correspond to
lighter fry colors. DBH = days before harvest.

| | ‘Ranger’ | 1632-1 | 1632-3 | 1632-4 | 1632-5 | 1632-21 |
|---|---|---|---|---|---|---|
| 35 days dbh | 12.7 | 24.5 | 19.5 | 33.1 | | 25.3 |
| 28 days dbh | 12.6 | 30.6 | 19.8 | 21.2 | | 17.9 |
| 21 days dbh | 26.5 | | | 29.9 | 33.6 | 35.0 |
| 14 days dbh | 22.7 | 46.1 | 44.8 | | 38.4 | |
| 7 days dbh | 22.7 | 47.3 | 45.2 | 42.0 | 36.7 | 46.7 |
| No infection | 32.6 | 49.2 | 42.3 | 37.9 | 39.9 | 44.0 |

### Example 3

### Polyphenol Oxidase Silencing does not Minimize the Symptoms Associated with Zebra Chip

Sense and antisense fragments of the Polyphenol oxidase-5 5′-UTR (Ppo5, SEQ ID NOs: 8 and 9), were arranged as inverted repeat between two convergent promoters—the ADP glucose pyrophosphorylase gene (Agp, SEQ ID NO: 6) and the promoter of the granule-bound synthase gene (Gbss, SEQ ID NO: 11) to induce silencing of the Ppo5 gene. The sense and antisense fragments of the Ppo 5′UTR were separated by non-coding spacer DNA (SEQ ID NO: 12). This method of gene silencing described previously (Yan et al. Plant Physiol. 141:1508-1518, 2006) ensures the silencing of the Ppo gene but any fragment of the Ppo gene down to 21-23 base pairs of the Ppo cDNA sequence could be used for silencing. The P-DNA vector and the marker-free method used to produce the intragenic lines F10, E12, and J3 is described previously (Rommens et al. Plant Biotechnol. J., 6:843-853, 2008).

Preparation and growth of the LBA4044 strain of Agrobacterium harboring the polyphenol oxidase silencing cassette proceeded as described in the previous examples. Potato transformation to generate Ppo silenced lines proceeded as described for the generation of invertase silenced lines in the previous example 1. The transcript levels of Ppo5 gene in tubers of untransformed plants and their intragenic counterparts were determined by Northern blot analysis (FIG. 5B). In greenhouse-grown tubers the transcription level of Ppo5 gene was strongly reduced in F10, E12 and J3 intragenic events compared to their untransformed controls, indicating that the Ppo5 gene was silenced in the modified tubers. We chose to silence Ppo in the ‘Atlantic’, Russet Burbank and ‘Ranger’ Russet varietal backgrounds. All three varieties are susceptible to blackspot bruise but when transformed with a Ppo silencing cassette, they show no susceptibility to blackspot bruise. This fact is illustrated for the ‘Atlantic’ wild-type and the Ppo-silenced equivalent in FIGS. 4A and 4B.

A field trial using greenhouse-grown seed for the untransformed controls, empty vector controls and Ppo-silenced lines was conducted at Texas A&M University Bushland Research and Extension Center in Bushland, Tex. as described for invertase-silenced lines above. The means of scoring the fresh ZC symptoms of Ppo-silenced lines and their respective controls is described above and summarized in Table 2. From these scores, it is apparent that Ppo silencing does not minimize the fresh symptom development in ZC-infected tubers in any of the three varietal backgrounds. The polyphenol oxidase-silenced lines scored no better than the untransformed controls, showing that fresh symptoms cannot be alleviated by the silencing of Ppo. More importantly, after frying the available lines according to methods described above in example 2, it is clear that Ppo silencing does not make ZC infected chips lighter. Agtron readings in Table 4 show that Ppo-silenced J3 is not lighter than the untransformed ‘Atlantic’ control. Such is true when comparing the E12 line to the Russet Burbank control or the F10 line to the ‘Ranger’ Russet control.

TABLE 4
Agtron readings for of chips prepared from ZC-infected and uninfected
tubers with and without polyphenol oxidase silencing. Each value
is an average of 3 readings on the same sample. Certain data points
are missing due to crop failure. Higher numbers correspond to
lighter fry colors. DBH = days before harvest.

| | J3 | ‘Atlantic’ | E12 | Burbank | F10 | ‘Ranger’ |
|---|---|---|---|---|---|---|
| 35 days dbh | 38.9 | 44.1 | 13.5 | 13.5 | 12.1 | 12.7 |
| 28 days dbh | 38.4 | 37.5 | 15.6 | 14.4 | | 12.6 |
| 21 days dbh | 37.2 | 36.8 | 28.3 | 28.5 | 26.7 | 26.5 |
| 14 days dbh | 45.3 | 45 | | 25.5 | 22.4 | 22.7 |
| 7 days dbh | 45.8 | 47.1 | | 27.9 | 22.9 | 22.7 |
| No infection | 48.3 | 49.2 | | 27.9 | 32.7 | 32.6 |

We confirmed the rapid browning response of cut or peeled, ZC-infected tubers (Navarre et al., Amer. J. Potato Res. 86:88-95 2009). Polyphenol oxidase silencing suppresses this reaction as visualized in FIG. 4. Neither uninfected (4B) nor infected (4C) Ppo-silenced tubers show the darkening.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A method of minimizing the frequency of sugar ends in potato tuber or products made from said potato tuber, comprising disrupting the vacuolar invertase enzyme activity in said potato tuber, wherein the frequency of sugar ends in the potato tuber is reduced in comparison to a control potato tuber.

2. A method of minimizing the symptoms of Zebra chip in potato tuber or products made from said potato tuber, comprising disrupting the vacuolar invertase enzyme activity in said potato tuber, wherein the symptoms of Zebra chip in the potato tuber is reduced in comparison to a control potato tuber.

3. The method of claim 1 or claim 2, wherein the vacuolar invertase enzyme activity is disrupted by introducing one or more nucleotide changes of the vacuolar invertase gene encoding the vacuolar invertase enzyme into the potato tuber.

4. The method of claim 1 or claim 2, wherein the vacuolar invertase enzyme activity is disrupted by introducing an inhibitory nucleotide sequence.

5. The method of claim 4, wherein the inhibitory nucleotide sequence is selected from the group consisting of antisense RNA sequences, dsRNAi sequences, and inverted repeats.

6. The method of claim 4, wherein the inhibitory nucleotide is operably linked to a plant promoter.

7. The method of claim 6, wherein the plant promoter is selected from the group consisting of constitutive promoters, non-constitutive promoters, inducible promoters, tissue specific promoters, and cell-type specific promoters.

8. The method of claim 7, wherein the tissue specific promoter is a tuber-specific promoter.

9. The method of claim 8, wherein the tuber-specific promoter is a promoter associated with an ADP glucose pyrophosphorylase gene.

10. The method of claim 9, wherein the tuber-specific promoter comprises the nucleic acid sequence SEQ ID NO: 6, or any functional variants therefore or functional fragments thereof.

11. The method of claim 5, wherein the inhibitory nucleotide sequence is an inverted repeat sequence.

12. The method of claim 11, wherein the inverted repeat is derived from SEQ ID NO: 5.

13. The method of claim 12, wherein the inverted repeat comprises a sense sequence corresponding to +53 to +733 of SEQ ID NO: 5.

14. The method of claim 13, wherein the inverted repeat comprises an anti-sense sequence corresponding to +552 to +49 of SEQ ID NO: 5.

15. The method of claim 12, wherein the inverted repeat comprises a sense sequence comprising a polynucleotide sequence selected from the group consisting of SEQ ID NOs: 3, 15, 16, and 18.

16. The method of claim 15, wherein the inverted repeat comprises an anti-sense sequence comprising a polynucleotide sequence selected from the group consisting of SEQ ID NOs: 4, 13, 14, 17, and 21.

17. The method of claim 1 or claim 2, wherein the method comprises expressing a gene silencing cassette in a potato plant, wherein the cassette comprises a sense sequence and an antisense sequence oriented as an inverted repeat, wherein the sense sequence has 100% identity to SEQ ID NO: 5 and the antisense sequence is a full length or partial reverse and complement sequence of the sense sequence.

18. The method of claim 17, wherein the sense sequence and the antisense sequence is separated by a spacer.

19. The method of claim 17, wherein the expression cassette comprises a tuber-specific promoter.

20. The method of claim 19, wherein the tuber-specific promoter is operably linked to the sense and the antisense sequences.

21. The method of claim 17, wherein the expression of cassette down-regulates the expression of at least one endogenous invertase gene thereby minimizing the frequency of sugar ends in potato tuber or products made from said potato tuber, and/or minimizing the symptoms of Zebra chip in potato tuber or products made from said potato tuber.

22. The method of claim 17, wherein the sense sequence is a full length or partial of SEQ ID NO: 5.

23. The method of claim 17, wherein the antisense sequence is 100% identical to the reverse and complement sequence of the sense sequence.

24. The method of claim 17, wherein the antisense sequence is not 100% identical to, but partially overlapped with the reverse and complement sequence of the sense sequence.


SEQUENCE LISTING (21 sequences)

SEQ ID NO: 1 - DNA, 28 bp, Artificial Sequence
  primer
cggatccacacattcctcccggatcaac

SEQ ID NO: 2 - DNA, 28 bp, Artificial Sequence
  primer
cgggcccagcggacccagtccagacacc

SEQ ID NO: 3 - DNA, 681 bp, Artificial Sequence
  sense fragment of potato acid invertase
acacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatataaacggtgtctggactgggtccgctaccatcctacccgatggtcagatcatgatgctttataccggtgacactgatgattatgtacaagtgcaaaatcttgcgtaccccactaacttatctgatcctctccttctagactgggtcaagtacaaaggcaacccggttctggttcctccacccggcattggtgtcaaggactttagag

SEQ ID NO: 4 - DNA, 504 bp, Artificial Sequence
  antisense fragment of potato acid invertase (504 bp)
agcggacccagtccagacaccgttaatatcgtaccattgatcaggaaccatggcaaaaggcaagtagagccagtggatcaagtccttggatacggcatggccccatgtgatatttccccaaatagctgaatctggattgtattgataaaaaagatgataccatcccttgtggtacaatggaccattaggatcgttcatccaatttttttgaggttgaaaatggtaagcagttctttgccagctaagcatagcattggaccacgcataagaaacgtgactagcattgacgacatctcgaaaagtcttatcggagactccctgagaaacacctcttgacggcggcgccggcgaacgggagttactctgcaagtccggtgactggttgttgaggatcggaaagaaggctacagaaagcaaaaggaaagaggagaggaaaatgccggagatgattttaagggacttccggtggccggaatcgggttgatccgggaggaatgtgtaatg

SEQ ID NO: 5 - DNA, 1920 bp, Solanum tuberosum
atggccacgcagtaccattccagttatgacccggaaaactccgcctcccattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatattaacggtgtctggactgggtccgctaccatcctacccgatggtcagatcatgatgctttataccggtgacactgatgattatgtacaagtgcaaaatcttgcgtaccccactaacttatctgatcctctccttctagactgggtcaagtacaaaggcaacccggttctggttcctccacccggcattggtgtcaaggactttagagacccgaccactgcttggaccggaccccaaaatgggcaatggctcttaacaatcgggtctaagattggtaaaacgggtattgcacttgtttatgaaacttccaacttcacaagctttaagctattggatgaagtgctgcatgcggttccgggtacgggtatgtgggagtgtgtggacttttacccggtatcgactgaaaaaacaaacgggttggacacatcatataacggcccgggtgtaaagcatgtgttaaaagcaagtttagatgacaataagcaagatcactatgctattgggacgtatgacttgacaaagaacaaatggacacccgataacccggaattggattgtggaattgggttgaagctggattatgggaaatattatgcatcaaagacattttatgacccgaagaaacaacgaagagtactgtggggatggattggggaaactgatagtgaatctgctgacctgcagaagggatgggcatctgtacagagtattccaaggacagtgctttacgacaagaagacagggacacatctacttcagtggccagttgaagaaattgaaagcttaagagcgggtgatcctattgttaagcaagtcaatcttcaaccaggttcaattgagctactccatgttgactcagctgcagagttggatatagaagcctcatttgaagtggacaaagtcgcgctccagggaataattgaagcagatcatgtaggtttcagctgctctactagtggaggtgctgctagcagaggcattttgggaccatttggtgtcgttgtaattgctgatcaaacgctatctgagctaacgccagtttacttcttcatttctaaaggagctgatggtcgagctgagactcacttctgtgctgatcaaactagatcctcagaggctccgggagttgctaaacgagtttatggtagttcagtacccgtgttggacggtgaaaaacattcgatgagattattggtggaccactcaattgtggagagctttgctcaaggaggaagaacagtcataacatcgcgaatttacccaacaaaggcagtgaatggagcagcacgactcttcgttttcaacaatgccacaggggctagcgtgactgcctccgtcaagatttggtcacttgagtcggctaatattcgatccttccccttgcaagacttgtaa

SEQ ID NO: 6 - DNA, 2260 bp, Solanum tuberosum
aagtgtctgagacaaccaaaactgaaagtgggaaaccaaactctaagtcaaagactttatatacaaaatggtataaatataattatttaatttactatcgggttatcgattaacccgttaagaaaaaacttcaaaccgttaagaaccgataacccgataacaaaaaaaatctaaatcgttatcaaaaccgctaaactaataacccaatattgataaaccaataactttttttattcgggttatcggtttcagttctgtttggaacaatcctagtgtcctaattattgttttgagaaccaagaaaacaaaaacttacgtcgcaaatatttcagtaaatacttgtatatctcagtgataattgatttccaacatgtataattatcatttacgtaataatagatggtttccgaaacttacgcttcccttttttcttttgcagtcgtatggaataaaagttggatatggaggcattcccgggccttcaggtggaagagacggagctgcttcacaaggagggggttgttgtacttgaaaatgggcatttattgttcgcaaacctatcatgttcctatggttgtttatttgtagtttggtgttcttaatatcgagtgttctttagtttgttccttttaatgaaaggataatatctgtgcaaaaataagtaaattcggtacataaagacatttttttttgcattttctgtttatggagttgtcaaatgtgaatttatttcatagcatgtgagtttcctctcctttttcatgtgcccttgggccttgcatgtttcttgcaccgcagtgtgccagggctgtcggcagatggacataaatggcacaccgctcggctcgtggaaagagtatggtcagtttcattgataagtatttactcgtattcggtgtttacatcaagttaatatgttcaaacacatgtgatatcatacatccattagttaagtataaatgccaactttttacttgaatcgccgaataaatttacttacgtccaatatttagttttgtgtgtcaaacatatcatgcactatttgattaagaataaataaacgatgtgtaatttgaaaaccaattagaaaagaagtatgacgggattgatgttctgtgaaatcactggtaaattggacggacgatgaaatttgatcgtccatttaagcatagcaacatgggtctttagtcatcatcattatgttataattattttcttgaaacttgatacaccaactttcattgggaaagtgacagcatagtataaactataatatcaattctggcaatttcgaattattccaaatctcttttgtcatttcatttcctcccctatgtctgcaagtaccaattatttaagtacaaaaaatcttgattaaacaatttattttctcactaataatcacatttaatcatcaacggttcatacacgtctgtcactctttttttattctctcaagcgcatgtgatcataccaattatttaaatacaaaaaatcttgattaaacaattcagtttctcactaataatcacatttaatcatcaacggttcatacacatccgtcactctttttttattctctcaagcgcatgtgatcataccaattatttaaatacaaaaaatcttgattaaacaattcattttctcactaataatcacatttaatcatcaacggtttatacacgtccgccactctttttttattctctcaagcgtatgtgatcatatctaactctcgtgcaaacaagtgaaatgacgttcactaataaataatcttttgaatactttgttcagtttaatttatttaatttgataagaatttttttattattgaatttttattgttttaaattaaaaataagttaaatatatcaaaatatcttttaattttatttttgaaaaataacgtagttcaaacaaattaaaattgagtaactgtttttcgaaaaataatgattctaatagtatattctttttcatcattagatattttttttaagctaagtacaaaagtcatatttcaatccccaaaatagcctcaatcacaagaaatgcttaaatccccaaaataccctcaatcacaagacgtgtgtaccaatcatacctatggtcctctcgtaaattccgacaaaatcaggtctataaagttacccttgatatcagtattataaaactaaaaatctcagctgtaattcaagtgcaatcacactctaccacacactctctagtagagagatcagttgataacaagcttgttaac

SEQ ID NO: 7 - DNA, 361 bp, Solanum tuberosum
ctagtttttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaatagg

SEQ ID NO: 8 - DNA, 144 bp, Artificial Sequence
  sense PPO trailer
tagtctctattgaatctgctgagattacactttgatggatgatgctctgtttttgttttcttgttctgtttttttatgttgaaatcagctttgttgcttgatttcattgaagttgttattcaagaataaatcagttacaattat

SEQ ID NO: 9 - DNA, 144 bp, Artificial Sequence
  antisense PPO trailer
ataattgtaactgatttattcttgaataacaacttcaatgaaatcaagcaacaaagctgatttcaacataaaaaaacagaacaagaaaacaaaaacagagcatcatccatcaaagtgtaatctcagcagattcaatagagacta

SEQ ID NO: 10 - DNA, 1958 bp, Solanum tuberosum
tcttttgcgttttgagcaataatggcaagcttgtgcaatagtagtagtacatctctcaaaactccttttacttcttcctccacttctttatcttccactcctaagccctctcaacttttcatccatggaaaacgtaaccaaatgttcaaagtttcatgcaaggttaccaataataacggtgaccaaaaccaaaacgttgaaacaaattctgttgatcgaagaaatgttcttcttggcttaggtggtctttatggtgttgctaatgctataccattagctgcatccgctgctccagctccacctcctgatctctcgtcttgtagtatagccaggattaacgaaaatcaggtggtgccgtacagttgttgcgcgcctaagcctgatgatatggagaaagttccgtattacaagttcccttctatgactaagctccgtgttcgtcagcctgctcatgaagctaatgaggagtatattgccaagtacaatctggcgattagtcgaatgaaagatcttgataagacacaacctttaaaccctattggttttaagcaacaagctaatatacattgtgcttattgtaacggtgcttatagaattggtggcaaagagttacaagttcataattcttggcttttcttcccgttccatagatggtacttgtacttccacgagagaatcgtgggaaaattcattgatgatccaactttcgctttaccatattggaattgggaccatccaaaaggtatgcgttttcctgccatgtatgatcgtgaagggacttcccttttcgatgtaacacgtgaccaaagtcaccgaaatggagcagtaatcgatcttggttttttcggcaatgaagttgaaacaactcaactccagttgatgagcaataatttaacactaatgtaccgtcaaatggtaactaatgctccatgtcctcggatgttctttggcgggccttatgatctcggggttaacactgaactcccgggaactatagaaaacatccctcacggtcctgtccacatctggtctggtacagtgagaggttcaactttgcccaatggtgcaatatcaaacggtgagaatatgggtcatttttactcagctggtttggacccggttttcttttgccatcacagcaatgtggatcggatgtggagcgaatggaaagcgacaggagggaaaagaacggatatcacacataaagattggttgaactccgagttctttttctatgatgaaaatgaaaacccttaccgtgtgaaagtcagagactgtttggacacgaagaagatgggatacgattacaaaccaattgccacaccatggcgtaacttcaagcccttaacaaaggcttcagctggaaaagtgaatacagcttcacttccgccagctagcaatgtattcccattggctaaactcgacaaagcaatttcgttttccatcaataggccgacttcgtcaaggactcaacaagagaaaaatgcacaagaggagatgttgacattcagtagcataagatatgataacagagggtacataaggttcgatgtgttttcgaacgtggacaataatgtgaatgcgaatgagcttgacaaggcggagtttgcggggagttatacaagtttgccacatgttcatagagctggtgagactaatcatatcgcgactgttgatttccagctggcgataacggaactgttggaggatattggtttggaagatgaagatactattgcggtgactctggtgccaaagagaggtggtgaaggtatctccattgaaggtgcgacgatcagtcttgcagattgttaattagtctctattgaatctgctgagattacactttgatggatgatgctctgtttttgttttcttgttctgttttttcctctgttgaaatcagctttgttgcttgatttcattgaagttgttattcaagaataaatcagttacaa

SEQ ID NO: 11 - DNA, 686 bp, Solanum tuberosum
gaaccatgcatctcaatcttaatactaaaaaatgcaacaaaattctagtggagggaccagtaccagtacattagatattatcttttattactataataatattttaattaacacgagacataggaatgtcaagtggtagcggtaggagggagttggttcagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcactcacacagctcaacaagtggtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagcttggtagattcccctttttgtagaccacacatcac

SEQ ID NO: 12 - DNA, 157 bp, Artificial Sequence
  Spacer1
gtgtatgggtgatccttctcttattataccgactaaagacattggtattaaggatatcttatcttttgaggagattcccgttcagattctggagcgtcaggttcgcaagttgagaaccaatgaggtaacatcagtcaaggtcttatggaggaatcag

SEQ ID NO: 13 - DNA, 636 bp, Artificial Sequence
  StInv (antisense in p2166 and p2168)
caagattttgcacttgtacataatcatcagtgtcaccggtataaagcatcatgatctgaccatcgggtaggatggtagcggacccagtccagacaccgttaatatcgtaccattgatcaggaaccatggcaaaaggcaagtagagccagtggatcaagtccttggatacggcatggccccatgtgatatttccccaaatagctgaatctggattgtattgataaaaaagatgataccatcccttgtggtacaatggaccattaggatcgttcatccaatttttttgaggttgaaaatggtaagcagttctttgccagctaagcatagcattggaccacgcataagaaacgtgactagcattgacgacatctcgaaaagtcttatcggagactccctgagaaacacctcttgacggcggcgccggcgaacgggagttactctgcaagtccggtgactggttgttgaggatcggaaagaaggctacagaaagcaaaaggaaagaggagaggaaaatgccggagatgattttaagggacttccggtggccggaatcgggttgatccgggaggaatgtgtaatgggaggcggagttttccgggtcataactggaatggtactgcgtggccatactcgtgc

SEQ ID NO: 14 - DNA, 122 bp, Artificial Sequence
  StInv trailer (antisense in p2166 and p2168)
ctaaaatggcgaataagtagagtataacactacatattctccctctcttccctttcttgatgggacatcggtgaaataaccttcaaatgaaaaaaagaatgaagaagatatggcttgatgaa

SEQ ID NO: 15 - DNA, 636 bp, Artificial Sequence
  StInv sense in p2166 and p2168
gcacgagtatggccacgcagtaccattccagttatgacccggaaaactccgcctcccattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatattaacggtgtctggactgggtccgctaccatcctacccgatggtcagatcatgatgctttataccggtgacactgatgattatgtacaagtgcaaaatcttg

SEQ ID NO: 16 - DNA, 122 bp, Artificial Sequence
  StInv trailer (sense in p2166 and p2168)
ttcatcaagccatatcttcttcattctttttttcatttgaaggttatttcaccgatgtcccatcaagaaagggaagagagggagaatatgtagtgttatactctacttattcgccattttag

SEQ ID NO: 17 - DNA, 402 bp, Artificial Sequence
  StInv (antisense in p2576)
ttgacccagtctagaaggagaggatcagataagttggtggggtacgcaagattttgcacttgtacataatcatcagtgtcaccggtataaagcatcatgatctgaccatcgggtaggatggtagcggacccagtccagacaccgtttatatcgtaccattgatcaggaaccatggcaaaaggcaagtagagccagtggatcaagtccttggatacggcatggccccatgtgatatttccccaaatagctgaatctggattgtattgataaaaaagatgataccatcccttgtggtacaatggaccattaggatcgttcatccaatttttttgaggttgaaaatggtaagcagttctttgccagctaagcatagcattggaccacgcataagaaacgtgacta

SEQ ID NO: 18 - DNA, 682 bp, Artificial Sequence
  StInv (sense in p2576)
gcacgagtatggccacccagtaccattccagttatgacccggaaaactccgcctcccattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatataaacggtgtctggactgggtccgctaccatcctacccgatggtcagatcatgatgctttataccggtgacactgatgattatgtacaagtgcaaaatcttgcgtaccccaccaacttatctgatcctctccttctagactgggtcaa

SEQ ID NO: 19 - DNA, 31 bp, Artificial Sequence
  primer
cgggcccctctaaagtccttgacaccaatgc

SEQ ID NO: 20 - DNA, 29 bp, Artificial Sequence
  primer
cactagtcattacacattcctcccggatc

SEQ ID NO: 21 - DNA, 681 bp, Artificial Sequence
  antisense fragment of potato acid invertase (681 bp)
ctctaaagtccttgacaccaatgccgggtggaggaaccagaaccgggttgcctttgtacttgacccagtctagaaggagaggatcagataagttagtggggtacgcaagattttgcacttgtacataatcatcagtgtcaccggtataaagcatcatgatctgaccatcgggtaggatggtagcggacccagtccagacaccgtttatatcgtaccattgatcaggaaccatggcaaaaggcaagtagagccagtggatcaagtccttggatacggcatggccccatgtgatatttccccaaatagctgaatctggattgtattgataaaaaagatgataccatcccttgtggtacaatggaccattaggatcgttcatccaatttttttgaggttgaaaatggtaagcagttctttgccagctaagcatagcattggaccacgcataagaaacgtgactagcattgacgacatctcgaaaagtcttatcggagactccctgagaaacacctcttgacggcggcgccggcgaacgggagttactctgcaagtccggtgactggttgttgaggatcggaaagaaggctacagaaagcaaaaggaaagaggagaggaaaatgccggagatgattttaagggacttccggtggccggaatcgggttgatccgggaggaatgtgt